(19) United States
(12) Plant Patent Application Publication
(10) Pub. No.: US 2026/0293787 P1
(43) Pub. Date: Sep. 24, 2026

(54) GEOGENANTHUS PLANT NAMED 'GEOFLAMAUR'

(50) ;

(71) Applicants: ANGELA RAPISARDA, BLEISWIJK (NL); LEENDERT JOHANNES VAN VLIET, BLEISWIJK (NL)

(72) Inventors: ANGELA RAPISARDA, BLEISWIJK (NL); LEENDERT JOHANNES VAN VLIET, BLEISWIJK (NL)

(73) Assignees: CASA BOTANICA, BLEISWIJK (NL); FLORALI LICENSING BV, BLEISWIJK (NL)

(21) Appl. No.: 19/571,089

(22) Filed: Mar. 18, 2026

(30) Foreign Application Priority Data
Mar. 18, 2025 (QZ) 2025/0493

(51) Int. Cl. A01H 6/00; A01H 5/12

(52) U.S. Cl. CPC A01H 6/00; A01H 5/12
USPC PLT/373

(57) ABSTRACT

A new and distinct Geogenanthus plant named ‘GEOFLAMAUR’ particularly distinguished by foliage displaying distinct, irregular striped variegation with yellow, green and greyed-purple colorations, young foliage often emerging green or occasionally yellow before developing the distinctive striped variegation pattern, and foliage coloration that changes as leaves mature, gradually becoming greener while retaining the distinctive striped leaf variegation.

Genus and species: Geogenanthus ciliatus.

Variety denomination: ‘GEOFLAMAUR’.

### STATEMENT REGARDING PRIOR DISCLOSURES BY THE INVENTOR

The Applicant asserts that no publications or advertisements relating to sales, offers for sale, or public distribution occurred more than one year prior to the effective filing date of this application. Any information about the claimed plant would have been obtained from a direct or indirect disclosure from the Inventor. The Applicant claims a prior art exemption under 35 U.S.C. 102(b) (1) for disclosures and/or sales that fall within a one-year grace period prior to the filing date.

### BACKGROUND OF THE INVENTION

The present invention comprises a new and distinct variety of Geogenanthus plant, botanically known as Geogenanthus ciliatus, and hereinafter referred to by the variety name ‘GEOFLAMAUR’. The new Geogenanthus cultivar ‘GEOFLAMAUR’ originated as a naturally occurring mutation discovered by the inventors growing amongst a cultivated population of unnamed and unpatented Geogenanthus ciliatus plants in a greenhouse located in Nootdorp, The Netherlands. During the years of 2021 and 2022, several naturally occurring mutations were selected by the inventors from said population of Geogenanthus ciliatus plants. The selected mutations exhibited variegated foliage in varying colors and patterns. These selections were subsequently asexually propagated and grown from March 2022 until October 2022 to determine agronomic qualities, stability, and commercial potential. ‘GEOFLAMAUR’ was selected in October 2022 from amongst said mutations due to its stable striped leaf variegation pattern and attractive foliage coloration.

The new Geogenanthus cultivar ‘GEOFLAMAUR’ was further asexually propagated beginning in November 2022 by meristem tissue culture in a laboratory located in Roelofarendsveen, The Netherlands. Since that time, ‘GEOFLAMAUR’ has been asexually propagated through tissue culture and has undergone at least seventeen cycles of propagation, during which the unique characteristics of the ‘GEOFLAMAUR’ have been observed to remain stable and reproduced true to type.

### SUMMARY OF THE INVENTION

The new Geogenanthus cultivar ‘GEOFLAMAUR’ has not been observed under all possible environmental conditions. The phenotype may vary somewhat with variations in environment such as temperature, day length, light intensity, water status, fertilizer rate and type, without, however, any variance in genotype.

The following are the most outstanding and distinguishing characteristics of this new Geogenanthus cultivar. The combination of these characteristics distinguishes ‘GEOFLAMAUR’ as a new and distinct variety of Geogenanthus.
1. ‘GEOFLAMAUR’ exhibits foliage displaying distinct, irregular striped leaf variegation with yellow, green and greyed-purple colorations.
2. ‘GEOFLAMAUR’ exhibits young foliage often emerging green or occasionally yellow before developing the distinctive striped variegation pattern.
3. ‘GEOFLAMAUR’ exhibits foliage coloration that changes as leaves mature, gradually becoming greener while retaining the distinctive striped leaf variegation.

### DESCRIPTION OF THE PHOTOGRAPHS

This new Geogenanthus cultivar is illustrated by the accompanying-colored photographs which show the overall appearance and distinct characteristics of the plant. The colors shown are as true as can be reasonably obtained by conventional photographic procedures. The photographs were taken in February 2026 from plants grown in 9-centimeter containers in a conventional greenhouse located in Berkel en Rodenrijs, The Netherlands. Plants in the photographs are 18 weeks old. Colors in the photographs may differ slightly from the color values cited in the botanical description which accurately describes the colors of the new variety.

FIG. 1. shows the overall plant form and foliage of ‘GEOFLAMAUR’.

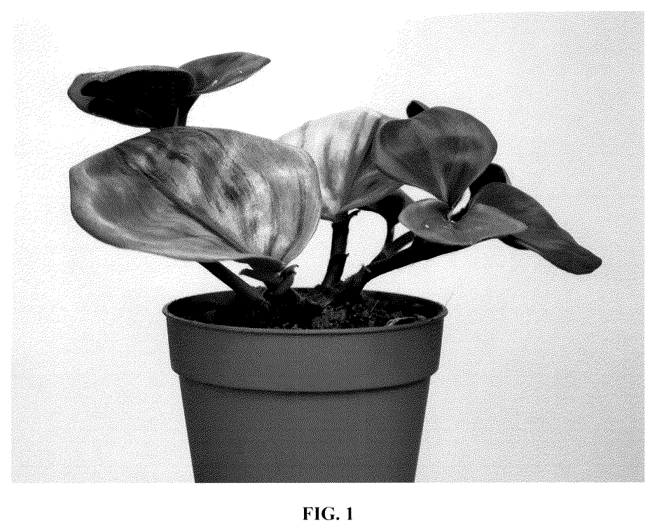

FIG. 2. shows a close-up of a typical mature leaf of ‘GEOFLAMAUR’.

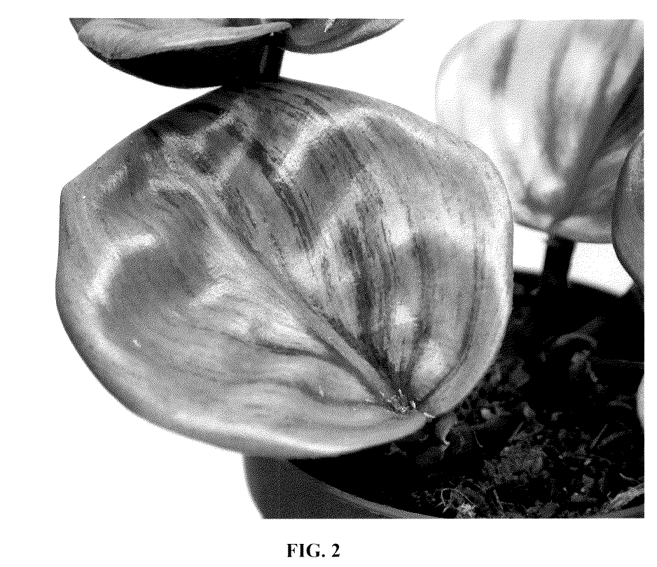

FIG. 3. shows a close-up of a typical juvenile leaf of ‘GEOFLAMAUR’.

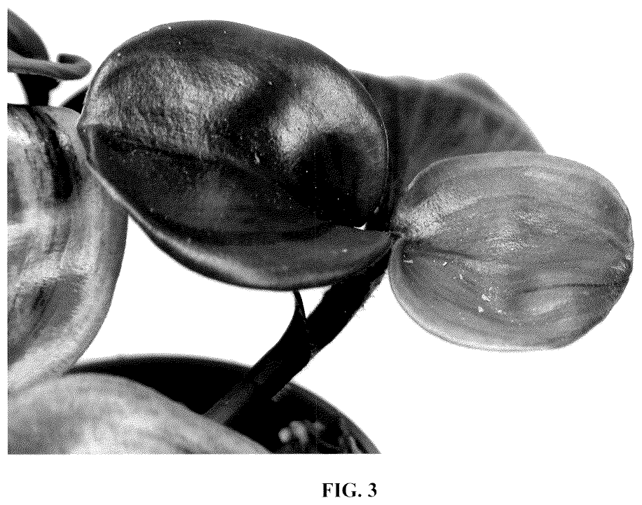

### DESCRIPTION OF THE NEW VARIETY

In the following description, color references are made to The Royal Horticultural Society Colour Chart, Sixth Edition, except where general color terms of ordinary dictionary significance are used.

The following observations and measurements describe 18-week-old plants grown in a conventional greenhouse located in Berkel en Rodenrijs, The Netherlands with night temperatures averaging 19° C. and day temperatures averaging 21° C. Plants were grown under normal daytime light levels with no artificial supplemental lighting provided. Detailed descriptions were taken in February 2026 from plants grown in 9-centimeter containers. Measurements and numerical values represent averages of typical plant types.

### DETAILED BOTANICAL DESCRIPTION

Classification:
   Family.—Commelinaceae.
   Botanical.—Geogenanthus ciliatus.
   Denomination.—‘GEOFLAMAUR’.
General description:
   Plant type.—Herbaceous perennial with short aerial stems bearing terminal rosettes of leaves.
   Approximate container size and/or cropping system.—7.0 cm or larger pots.
   Growth habit.—Broad spreading to spreading and upright.
   Overall plant shape.—Flattened.
   Height from soil level to top of foliar plane.—8.7 cm.
   Plant diameter or area of spread.—14.3 cm.
Stems:
   Branching characteristics.—Short, unbranched aerial stems arise directly from the soil and bear terminal rosettes of leaves.
   Quantity.—Average 5.
   Length.—Average 4.9 cm.
   Diameter.—Average 4.1 mm.
   Internode length.—Average 2.4 cm.
   Appearance and shape.—Circular.
   Luster.—Glossy.
   Stem aspect/angle from vertical.—Average 57.5°.
   Stem color.—Developing stems: N79B (purple). Mature stems: N186C (greyed-purple). Internodes: Close to N187A (greyed-purple).
   Pubescence.—Not present.
Foliage description:
   Arrangement.—Alternate, simple.
   Quantity of leaves per stem.—Average 3.
   Lamina.—Shape: Orbicular. Aspect: Slightly concave. Length: Average 4.6 cm. Width: Average 4.8 cm. Apex shape: Rounded with an abruptly acute tip. Base shape: Cordate. Margins: Slightly undulate with a revolute edge. Texture: Upper surface: Glabrous, smooth and moderately leathery. Lower surface: Glabrous, smooth and slightly leathery. Pubescence: Present; average length of hairs less than 0.1 mm and colored 164D (greyed-orange). Rugosity: Both sides non-rugose. Luster: Upper surface: Moderately glossy. Lower surface: Matte. Color: General color description: Juvenile foliage may initially appear yellow, depending on environmental conditions. As leaves mature, a striping pattern develops consisting of green, yellow and greyed-purple. Mature leaves gradually become greener, although the striping pattern may remain visible. The pattern and intensity of the striped leaf variegation may vary depending on environmental conditions such as light levels and temperature, and individual leaves may display slightly different striping patterns. Juvenile foliage: Upper surface: 139A (green) and striped with 147A (yellow-green). Lower surface: N186C (greyed-purple) and striped with a blend of N187A (greyed-purple) and 203A (black) with edges tinged N199A (greyed-brown). Mature foliage: Upper surface: N144A (yellowish green) and 151A (greenish yellow) and axially striped irregularly with 137C (green), 139A (green) and 147A (green); as leaves age, transitioning to 151A (greenish yellow). Lower surface: N77A (purple) and axially striped irregularly with a blend of N186B and N186C (greyed-purple); as leaves age, transitioning to 59B (red-purple) and axially striped irregularly with N186C (greyed-purple). Venation: Pattern: Parallel. Color (juvenile foliage, primary vein): Upper surface: N186B (greyed-purple). Lower surface: N79B (Purple). Color (mature foliage, primary vein): Upper surface: N186C (greyed-purple) and 187B (greyed-purple) towards the petiole; as leaves age, transitioning to 59A (red-purple). Lower surface: N78A (purple); as leaves age, transitioning to N186C (greyed-purple).
   Petiole.—Length: 0.6 cm. Shape and diameter: U-shaped, average 0.25 cm horizontally and vertically. Texture: Glabrous, smooth and moderately leathery. Luster (both upper and lower surfaces): Matte. Color (both upper and lower surfaces): 79B (purple).
   Stem bracts.—Shape: Ovate. Length: Average 1.3 cm. Width: Average 1.0 cm. Apex: Obtuse. Base: Sheathing. Margin: Entire. Pubescence: Short hairs approximately 1.5 mm in length and colored 164D (greyed-orange). Color: Upper surface: 147A (yellow-green) with N186C (greyed-purple) in the center. Lower surface: N189A (greyed-green).
Heat tolerance: At least tolerant to temperatures up to 40 degrees Celsius.
Cold tolerance: At least hardy to USDA zone 10.
USDA hardiness zones: Zones 10 to 13.
Disease and pest tolerance: No claims are made to any superior disease or insect resistance or tolerance.

### COMPARISON WITH THE PARENT PLANT

The new Geogenanthus cultivar ‘GEOFLAMAUR’ can be distinguished from its parent Geogenanthus ciliates plant in the following characteristics:
1. Plants of ‘GEOFLAMAUR’ display distinct striped variegated foliage, consisting of yellow, green and greyed-purple colorations which later transition into darker green tones as the leaves mature, whereas plants of the parent typically have uniform foliage coloration, which is generally very dark purple, and does exhibit striped variegation.
2. Leaves of ‘GEOFLAMAUR may initially appear yellow when young, particularly depending on environmental conditions, and gradually develop striped leaf variegation patterns as the leaves mature.

### COMPARISON WITH RELATED KNOWN VARIETY

The new Geogenanthus cultivar ‘GEOFLAMAUR’ can be distinguished from the related known variety of Geogenanthus cultivar ‘HGV15’ (unpatented) in the following characteristics:
1. Plants of ‘GEOFLAMAUR’ exhibit stronger and more stable striped leaf variegation, with consistent expression of yellow, green and greyed-purple colorations, whereas plants of ‘HGV15’ generally show less stable and less distinct variegation and the foliage color pattern is typically less pronounced.
2. The striped leaf variegation pattern of ‘GEOFLAMAUR’ is more clearly defined along the leaf blade, whereas leaves of ‘HGV15’ typically display less organized or weaker color patterns.

what is claimed is:

1. A new and distinct cultivar of Geogenanthus plant named ‘GEOFLAMAUR’, substantially as illustrated and described herein.
